(19) United States
(12) Patent Application Publication
(10) Pub. No.: US 2026/0314977 A1
(43) Pub. Date: Oct. 15, 2026

(54) SYSTEM AND METHOD FOR TESTING A CATHETER ELECTRODE ARRANGEMENT

(71) Applicant: BIOSENSE WEBSTER (ISRAEL) LTD., Yokneam (IL)

(72) Inventors: Shimon Miara, Haifa (IL); Shemer Shmaryau Berkowitz, Binyamina (IL)

(21) Appl. No.: 19/179,282

(22) Filed: Apr. 15, 2025

(51) Int. Cl. A61B 5/287; A61B 5/00; A61B 18/00

(52) U.S. Cl. CPC A61B 5/287; A61B 5/6858; A61B 5/6859; A61B 5/6869; A61B 18/00; A61B 2018/0016; A61B 2018/0022; A61B 2018/00351

(57) ABSTRACT

A method for testing a catheter carrying an electrode arrangement and a respective system are described. The method comprises: bringing a portion of the catheter having electrodes thereon, into contact with an electrode testing surface having a predetermined impedance per unit area and performing electrical impedance mapping of the electrode arrangement. The electrical impedance mapping comprising: selecting a pair of electrodes comprising at least one electrode of the electrode arrangement, closing an electrical circuit between the selected pair of electrodes, determining an impedance value of the electrical circuit, and determining electrical continuity of the pair of electrodes and a distance between the pair of electrodes on the electrode testing surface based on the impedance value. The method thus enables providing output data indicating electrical continuity and spatial mapping of the electrode arrangement based on the electrical impedance mapping.

### TECHNOLOGICAL FIELD

The present disclosure relates to testing catheters and specifically relates to testing spatial arrangement and electrical properties of an electrode arrangement of a catheter.

### BACKGROUND

As technology advances, the demand for more mechanically complex catheters with increased electrode numbers continues to grow. Modern catheters, particularly those used in medical procedures such as cardiac ablation, neurostimulation, and electrophysiology, may range from simple designs with basic electrode arrangements to more complex three-dimensional structures that enhance functionality and adaptability within the human body. These complex structures enable better navigation through anatomical pathways and more precise delivery of therapeutic interventions. However, the increased complexity of these catheters also introduces significant challenges in quality control and testing.

Testing such catheters requires the development of specialized testing equipment capable of accurately evaluating the performance and integrity of each electrode. Traditional testing methods often involve mechanical structures designed specifically for each catheter model. These structures generally need to ensure direct contact between each tested electrode and a dedicated test pin or pad that is precisely aligned with the catheter's electrode positions. For catheters with a three-dimensional design, achieving this precise alignment is difficult and often requires custom-built testers, increasing costs and development time.

### OVERVIEW

The present disclosure provides a method and a corresponding system for testing catheters carrying an electrode arrangement. The technique of the present disclosure utilizes a testing surface, or sheet, having selected electrical properties allowing for direct testing of properties of the catheter. Generally, testing a catheter according to the present disclosure can assess the catheter structures and electrodes arrangement, and provide indication on electrical continuity of various electrodes of the electrode arrangement. The test can provide further indications relating to the status and contact quality of the electrode arrangement.

More specifically, the technique of the present disclosure utilizes an electrode testing surface with a predetermined impedance per unit area for testing the catheter's electrode arrangement. For testing of a catheter, the method of the present disclosure involves positioning a portion of the catheter in contact with the electrode testing surface and performing electrical impedance mapping the electrode arrangement of the catheter. By selecting pairs of electrodes, closing an electrical circuit, and measuring impedance (e.g., resistance) values through the electrical circuit, the technique of the present disclosure can provide output data that indicates both the electrical continuity and the spatial positioning of the electrodes. This approach ensures effective testing of the operational status of the catheter's electrodes, allowing for reliable quality control and maintenance. In some examples, the electrode testing surface may be formed of a sheet of a selected material having a predetermined surface resistivity.

The technique of the present disclosure provides an ability to handle different types of catheters, including those with linear or multi-strand or multi-spine designs. For catheters with multiple strands, the strands may be spread in a star-like formation on a planar electrode testing surface to ensure proper electrical contact. Additionally, the electrode testing surface may be shaped into curved surfaces such as cylinders, cones, spheres, or ellipsoids, enabling direct testing of catheters with three-dimensional structures. This adaptability enhances the versatility of the testing method and system, enabling accurate testing across a wide range of catheter designs.

In some examples, the present disclosure provides a system for testing a catheter.

The system may comprise a electrode testing surface, an electrical controller circuit adapted to connect to a catheter and to an electrode arrangement thereof, an impedance measurement circuit adapted to determine impedance values between pairs of electrodes connected via the electrode testing surface, and a processing circuitry (e.g., one or more processors) configured and operable to receive impedance values measured between pairs of electrodes and determine one or more parameters of the electrode arrangement. In this connection, the processing circuitry can assess the catheter structure and arrangement of the electrode arrangement thereof. Further, the processing circuitry can determine data on electrical continuity of the electrodes, and optionally one or more electrical properties thereof.

For example, the system may also include a processing circuitry configured and operable to assess electrical continuity of the electrodes in electrode arrangement.

Further, the processing circuitry may also operate to determine a spatial map of the electrode arrangement, indicating relative locations of the electrodes on the electrode testing surface. Determining spatial mapping of the electrodes may be particularly beneficial in identifying possible faults or inconsistencies in the electrode pattern, contributing to improved safety and performance of catheters used in medical procedures. By providing both quantitative impedance measurements and qualitative spatial data, the disclosure offers a comprehensive solution for catheter testing and inspection.

The technique of the present disclosure thus provides a simple and efficient way for assessing properties of a catheter. The technique enables determining electrical conductivity and continuity thereof along the electrodes of the catheter, and may also provide data on relative locations of the electrodes, allowing to assess structural properties of the catheter.

Additionally, according to some examples, the method of the present disclosure may comprise first and second testing configurations, enabling to determine data on curvature of the catheter. For example, in a first testing configuration, the catheter may be placed to be in contact with the electrode testing surface, while being aligned to be straight, and conductivity of the electrodes may be tested. As indicated above, impedance variations can be used to estimate distances between the electrodes on the electrode testing surface. In a second testing configuration, the catheter may be placed in contact with the electrode testing surface, while being in its natural curvature. The electrodes are tested again to determine impedance between them along the electrode testing surface. Variation in distances between the electrodes provide data of curvature of the catheter in accordance with the electrodes' spatial arrangement.

### BRIEF DESCRIPTION OF THE DRAWINGS

In order to better understand the subject matter that is disclosed herein and to exemplify how it may be carried out in practice, examples will now be described, by way of non-limiting example only, with reference to the accompanying drawings, in which:

FIG. 1 illustrates a catheter-based electrophysiology mapping and ablation system, according to examples of the presently disclosed subject matter;

FIG. 2 schematically illustrates testing a catheter, and a catheter testing system according to some examples of the present disclosure;

FIG. 3 shows a general process of testing a catheter according to some examples of the present disclosure;

FIG. 4 exemplifies a process of mapping an electrode arrangement of a catheter according to some examples of the present disclosure;

FIG. 5 exemplifies determining relative locations of electrodes using triangulation according to some examples of the present disclosure;

FIGS. 6A to 6D exemplify different configurations of distal ends of catheters that can be tested using the technique according to some examples of the present disclosure; and

FIGS. 7A to 7C exemplify different configurations of the electrode testing surface according to some examples of the present disclosure.

### DETAILED DESCRIPTION

In the following detailed description, numerous specific details are set forth in order to provide a thorough understanding. However, it will be understood by those skilled in the art that the presently disclosed subject matter may be practiced without these specific details. In other instances, well-known methods and features have not been described in detail so as not to obscure the presently disclosed subject matter.

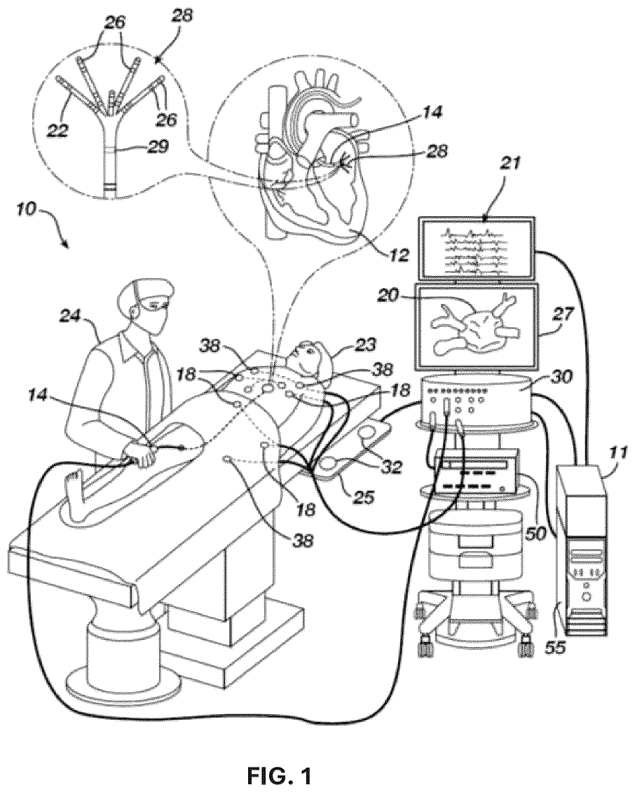

Reference is made to FIG. 1 showing an example catheter-based electrophysiology mapping and ablation system 10. System 10 includes at least one catheter, which is inserted by a physician 24 to the heart 12. Typically, a delivery sheath is inserted in an intrathoracic approach and guided to a desired location in heart 12, optionally via the apex of the heart. Thereafter, the catheter may be inserted into the delivery sheath catheter so as to arrive at the desired location in heart 12. In some examples, the desired location is the epicardium 71, and the catheter is introduced through the pericardium 73 so as to reach the epicardium.

An example catheter 14 that is configured for ablating tissue and/or for sensing electrical cardiac activity and/or mapping is illustrated herein. Physician 24 may place a distal end 28 of catheter 14 in contact with the epicardial tissue surface for sensing and/or ablating.

Catheter 14 is an exemplary catheter that includes a distal assembly, having one and preferably multiple electrodes 26 optionally distributed over a plurality of frame elements 22 at distal tip 28. The electrodes are generally configured for delivering ablation energy to tissue, and/or for sensing cardiac electrical signals. Catheter 14 additionally includes one or more position sensors 29 embedded in or near distal end 28 for tracking position and orientation of distal tip 28. Optionally and preferably, position sensor 29 is a magnetic based position sensor, for example a position sensor including three magnetic coils for sensing three-dimensional (3D) position and orientation; or a position sensor including one magnetic coil, for sensing a single direction.

Each of the magnetic based position sensors 29 may be operated together with a location pad 25 including a plurality of magnetic coils 32 configured to generate magnetic fields in a predefined working volume. Real time position of distal tip 28 of catheter 14 may be tracked based on magnetic fields generated with location pad 25 and sensed by magnetic based position sensor 29. Details of the magnetic based position sensing technology are described in U.S. Pat. Nos. 5,5391,199; 5,443,489; 5,558,091; 6,172,499; 6,239,724; 6,332,089; 6,484,118; 6,618,612; 6,690,963; 6,788,967; 6,892,091.

System 10 includes one or more electrode patches 38 positioned for skin contact on patient 23 to establish location reference for location pad 25 as well as impedance-based tracking of electrodes 26. For impedance-based tracking, electrical current is directed to electrodes 26 and sensed at electrode skin patches 38 so that the location of each electrode can be triangulated via the electrode patches 38. Details of the impedance-based location tracking technology are described in U.S. Pat. Nos. 7,536,218; 7,756,576; 7,848,787; 7,869,865; and 8,456,182.

A recorder 11 records and displays electrograms 21 captured with body surface ECG electrodes 18 and optionally cardiac signals captured with electrodes 26 of catheter 14. Recorder 11 may include pacing capability for pacing the heart rhythm and/or may be electrically connected to a standalone pacer.

System 10 may include an ablation energy generator 50 that is adapted to conduct ablative energy to electrodes 26 of the distal assembly at a distal tip of the catheter. Energy produced by ablation energy generator 50 may include, but is not limited to, radiofrequency (RF) energy or pulsed-field ablation (PFA) energy, including monopolar or bipolar high-voltage DC pulses as may be used to effect irreversible electroporation (IRE), or combinations thereof.

Patient interface unit (PIU) 30 is an interface configured to establish electrical communication between the at least one catheter with other catheters, other electrophysiological equipment, power supply and a workstation 55 for controlling operation of system 10. Electrophysiological equipment of system 10 may include for example, multiple catheters, location pad 25, body surface ECG electrodes 18, electrode patches 38, ablation energy generator 50, and recorder 11. Optionally and preferably, PIU 30 additionally includes processing capability for implementing real-time computations of location of the catheters and for performing ECG calculations.

Workstation 55 includes memory, processor unit with memory or storage with appropriate operating software stored therein, and user interface capability. Workstation 55 may provide multiple functions, optionally including (1) modeling the endocardial anatomy in three-dimensions (3D) and rendering the model or anatomical map 20 for display on a display device 27, (2) displaying on display device 27 activation sequences (or other data) compiled from recorded electrograms 21 in representative visual indicia or imagery superimposed on the rendered anatomical map 20, (3) displaying real-time location and orientation of the at least one catheter within the heart chamber, and (4) displaying on display device 27 sites of interest such as places where ablation energy has been applied. One commercial product embodying elements of the system 10 is available as the CARTO™ 3 System, available from Biosense Webster, Inc., 31A Technology Drive, Irvine, CA 92618.

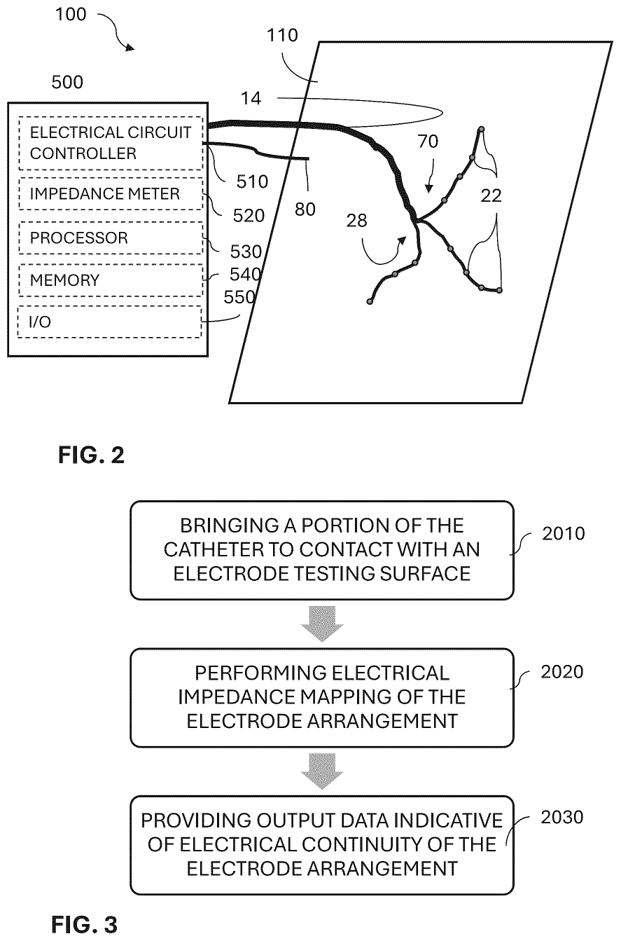

The above-described catheter-based electrophysiology mapping and ablation system 10, as well as many other systems utilizing one or more catheters equipped with an electrode arrangement at distal end 28 thereof may require testing, for ensuring electrical conductivity between electric contacts at the proximal end of the catheter and the electrodes at the distal end 28 thereof. Accordingly, and as described above, the present disclosure provides a method, and a corresponding system for testing one or more catheters carrying a respective electrode arrangement. Reference is made to FIG. 2 exemplifying a system 100 according to some examples of the present disclosure and illustrating testing of a catheter 14 in accordance with a method according to some examples of the present disclosure. The system and method of the present disclosure are based on the use of an electrode testing surface, or surface, 110 having a predetermined impedance per unit area. The sheet may be formed of a polymeric, ceramic or any other material, having certain characterizing impedance per unit area.

As illustrated in FIG. 2, according to some examples of the present disclosure, testing of a catheter 14 may involve bringing a portion, including frame elements 22 of the catheter into contact with the electrode testing surface 110. For example, in a catheter 14 having a plurality of strands, or frame elements 22, at the distal end thereof, the different strands 22 are to be spread on the surface of the electrode testing surface 110. In other examples, utilizing a distal end of a catheter having various different spatial structures, the electrode testing surface may be rolled or folded to provide desired contact of the electrode testing surface 110 and frame elements 22 of the distal end 28 of the catheter 14. Typically, the distal end 28 of the catheter 14 includes an electrode arrangement 70, generally including a plurality of electrodes 26. To enable proper testing of the catheter, the distal end 28 and the frame elements 22 thereof are preferably arranged on the electrode testing surface 110 to provide contact of the different electrodes 26 of the electrode arrangement 70 with the electrode testing surface 110.

The proximal end of the catheter may be connected to a controller 500 for performing testing of the catheter 14. The controller 500 may be configured as an electronic control circuit, connectable to a plurality of electrode lines, and for selectively close circuit using different electrode line for testing electrical conductivity and general impedance testing of such close circuits. The testing may utilize DC voltage for testing resistance through the circuits, or in some examples, the testing may utilize AC voltage or current enabling to further determine data on capacitance and inductance through the circuit. The controller 500 may further include one or more processors and memory, capable of executing desired computer readable instructions as listed herein below, for performing the catheter testing according to the various examples of the present disclosure.

In this connection, the controller 500 is exemplified in FIG. 2 including different modules, which may be software and/or hardware modules, the different modules are described herein in accordance with their respective functional operation and may be replaced by various elements as known in design of electronic devices. More specifically, controller 500 may include an electrical circuit controller 510, configured for providing electrical connection to the different electrodes of the electrode arrangement 70, typically via dedicated connections at the proximal end of the catheter. The electrical circuit controller 510, may generally enable selective connection to different electrodes and selecting pairs of electrodes to form an electrical circuit between the electrodes and the electrode testing surface 110. The controller 500 may further include: an impedance meter 520, configured for detection of impedance including resistance and optionally also capacitance and inductance, along an electrical circuit. Further, in some examples, the controller 500 may also include one or more processors 530, memory 540 and input/output communication module 550.

After bringing a portion, typically the distal end 28 which may include frame elements 22, of the catheter 14 into contact with the electrode testing surface 110, and connecting the proximal end to the controller 500, the present disclosure provides for performing testing of the electrode arrangement 70 of the catheter 14. As the different electrodes 26 are in contact with an electrode testing surface 110, testing may include closing circuits between different electrodes 26 through the catheter 14 and the electrode testing surface 50, and monitoring electrical properties of the closed circuits, and determining impedance measurement between pairs of electrodes 26. More specifically, the controller 500 may operate to select a pair of electrodes, close an electrical circuit between the electrodes of the selected pair, provide a selected voltage, or transmit a selected current through the circuit, and determine impedance through the circuit. Using direct current (DC) voltage or current, the impedance may be determined as resistance in accordance with Ohm's law V=IR. In some examples using alternating current (AC) voltage or current, the impedance Z may include resistance, capacitance and inductance, and may be determined in accordance with frequency response by v=iZ. Alternatively, or additionally, the impedance Z may be determined in accordance with a resonance frequency of the circuit and respective Q factor.

After determining impedance value in a closed circuit between a first pair of electrodes, the controller 500 may proceed to other pairs of electrodes, determining impedance values for different pairs of electrodes until sufficient data is collected. Sufficient data may include a condition where a selected number of electrodes have been tested, all electrodes have been tested, or in some examples, that the number of pairs of electrodes tested provides that each electrode has been tested within at least two pairs. After the testing is complete, the controller 500, e.g., using the input/output modules 550, may generate an output signal indicating test results and including data on electrical arrangement of the electrodes 26 in the catheter 14. The output data may generally include an indication that all tested electrodes provide electrical conductivity between the proximal and distal ends thereof. In some examples, the output data may further include data on electrical properties of the electrodes. Further, in some examples of the present disclosure, the output data may include spatial mapping of the electrode arrangement spread on the electrode testing surface. Such spatial data may be determined in accordance with varying impedance along different electrical circuits being tested, in accordance with typical impedance of the electrode testing surface 110.

The electrode testing surface 110 may be any sheet material having predetermined typical dielectric parameters, i.e., a dielectric sheet. For example, the electrode testing surface 110 may be formed of a polymeric surface/sheet having a predetermined resistance per unit area, or per unit length, of the sheet. For example, the electrode testing surface may be formed of polymers of ethylene plates, sheets or tape, such as XVCF-3S500 available from Caplinq. The electrode testing surface 110 has a surface resistivity of 50 Ω/square or greater, or 100 Ω/square or greater, 200 Ω/square or greater, 300 Ω/square or greater, or 400 Ω/square or greater, or 500 Ω/square or greater, to provide sufficient resistance through a closed circuit that includes a pair of electrodes connected through the electrode testing surface. For example, XVCF-3S500 sheet is typically characterized by surface resistivity of 500 Ω/square.

Further, in some examples, the controller circuit 500 may include an additional reference electric contact 80, configured to be in contact with the electrode testing surface 110 at a selected location. The controller may utilize the reference electrical contact 80 in combination with an additional electrode 26 of the electrode arrangement 70, forming pairs of electrodes. In this connection the controller may determine impedance values between electrodes 26 of the electrode arrangement 70 and the reference electrical contact 80. And to further utilize the determined impedance values to determine a spatial location of the electrodes 26 based on the impedance values. For example, the spatial locations of the electrodes based on the impedance values may be determined to be at a location (x,y) satisfying the relation:

$$R ( r ) = \frac{{R}_{sheet}}{2 π} \times ln [ \frac{r}{{r}_{0}} ]$$

where R(r) is the impedance value between the electrode through the reference electrical contact, Rₛₕₑₑₜ is the predetermined resistivity per unit area of the electrode testing surface, r is the distance between the electrode and the reference electrical contact, and r₀ is the radius of electrode touching contact area between the sheet and the electrode, and “ln” represents the natural logarithm.

Generally, in accordance with some examples of the present disclosure, the controller 500 and electrode testing surface 110 may together constitute a system for testing catheters 14 and the respective electrode arrangements 70. The system may be provided together with a set of instructions, printed or provided in electronic format. The instructions include an indication to an operator to bring the electrodes of the catheter to contact with the electrode testing surface 110, by spreading the frame elements 22 of distal end 28 of the catheter on the sheet, inserting the distal end 28 into a cavity within the rolled sheet etc. The instructions may also indicate a way of connecting the catheter 14 to the controller 500 and operating the controller to perform the selected testing procedure.

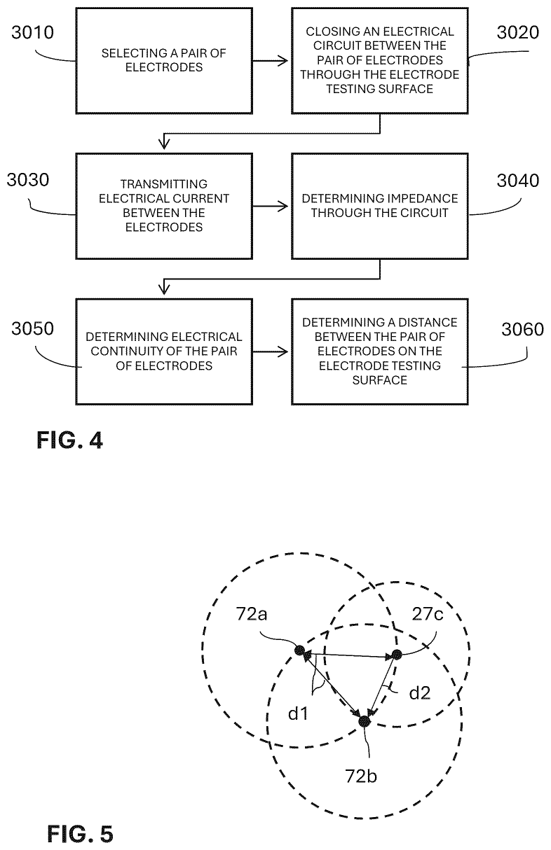

FIGS. 3 and 4 exemplify methods for testing an electrode arrangement and a catheter according to some examples of the present disclosure. FIG. 3 exemplifies testing electrical properties of a catheter according to some examples of the present disclosure, and FIG. 4 exemplifies testing electrical properties and spatial arrangement of the electrodes of a catheter according to some examples of the present disclosure. As shown in FIG. 3, the method includes bringing a portion, typically a distal end, of a catheter to contact with an electrode testing surface (step 2010). After the electrodes of the electrode arrangement are in contact with the electrode testing surface, the method utilizes conductivity through the electrode testing surface to perform electrical impedance mapping of the electrode arrangement (step 2020). The electrical impedance mapping generally includes determining impedance values for circuits that include different pairs of electrodes. For each pair of electrodes, the circuit includes the first and second electrodes of the pair, a portion of the electrode testing surface associated with shortest distance between the locations of the first and second electrodes on the electrode testing surface, and any electronic elements of the controller 500. Following electrical impedance mapping of the electrodes, the method includes generating output data indicative of electrical continuity of the electrodes (step 2030). Such output data may indicate if one or more of the electrodes is broken and does not provide electrical conductivity between proximal and distal ends of the catheter. Additionally, in some examples, the output data may include data on spatial arrangement of the electrodes.

Further, FIG. 4 exemplifies the electrical operations for testing an electrode arrangement and a catheter according to some examples of the present disclosure in more detail. This is generally performed after bringing the distal end, and the electrode arrangement of the catheter into contact with the electrode testing surface. As shown, the method includes selecting a pair of electrodes (step 3010), and closing an electrical circuit between the pair of electrodes through the electrode testing surface (step 3020). The electrical circuit generally includes a power source (e.g., a battery or more, or a power supply associated or connected to the controller), certain electronic elements including e.g., switches, resistors, voltmeters and/or amperemeter, also associated with the controller, and the electrical wires connecting between proximal end of the catheter and the active region of the electrodes at the distal end of the catheter. To determine electrical properties, the method further includes transmitting electrical current between the electrodes (step 3030) and determining impedance through the circuit (step 3040). In this connection, the power source may operate as a current source or a voltage source, i.e., either transmitting a selected current and measuring the voltage required for the current to flow or providing a selected voltage and measuring the current the flows in response.

For DC voltage/current, the impedance may be determined in accordance with Ohm's law (V=IR). In some examples, where the electrode arrangement includes additional electrical elements that require AC characterization, the impedance may be determined including capacitance and inductance based on a time varying current (or frequency response) in accordance with v=iZ, where v and I are the time varying voltage and current, and Z is the complex representation of the impedance. In accordance with transmission of electrical current through the closed circuit, the technique can determine electrical continuity of the selected pair of electrodes (step 3050). This is an initial testing criterion for testing the catheter. Further, based on data on surface resistivity of the electrode testing surface and the resistance determined in the circuit (being the resistance R, or real part of the impedance Z), the technique can estimate or determine a distance between the pair of electrodes on the electrode testing surface (step 3060). For example, the distance between the electrodes may be determined in accordance with

$$l = \frac{R \times W}{{R}_{sheet}}$$

where R is the determined resistance (or real part of the impedance Z) between the electrodes, Rₛₕₑₑₜ is the surface resistivity of the electrode testing surface, and W is the width of the electrodes.

The ability to determine a distance between pairs of electrodes when placed on the electrode testing surface enables spatial mapping of the electrode arrangement using some examples of the present disclosure. The general mapping technique may be associated with triangulation techniques for determining relative locations based on distance measurements. FIG. 5 exemplifies three electrodes 26a-26c and respective distances between them, enabling to determine spatial mapping of the electrodes on the electrode testing surface. As shows in FIG. 5, electrodes 26a and 26b are determined to be at certain distance d1 between them. Electrode 26c is at distance d1 (or any other distance) from electrode 26a, and at distance d2 from electrode 26b. Analyzing the distances and determining circles having respective radii enables mapping of the electrodes to determine their spatial location. While this is exemplified in FIG. 5 using three electrodes, this technique is suitable for any number of electrodes and enables checking for accuracy the larger the number of electrodes used.

In some examples, the technique of the present disclosure may utilize one or more reference electrodes positioned at known, predetermined, locations of the electrode testing surface. Such one or more reference electrodes may be used to improve determining spatial locations of the electrodes. For example, the reference electrodes may provide for eliminating or reducing ambiguities is electrode locations, or in examples where the electrode testing surface is not a planar sheet (e.g., folded or rolled to a three-dimensional shape such as a cone, cylinder, sphere, etc. When using reference electrodes, the reference electrodes may be selected to be within some of the pairs of electrodes, thus providing data on conductance of separate electrodes and providing data on distance between the known location of the electric contacts associated with the reference electrodes and other electrodes of the catheter. In this connection, determining a spatial locations of one or more electrodes based on the impedance values with respect to reference electric contacts having a known location may include determining a location (x,y) that satisfies the relation:

$$R ( r ) = \frac{{R}_{sheet}}{2 π} \times ln [ \frac{r}{{r}_{0}} ]$$

where R(r) is the impedance value between the electrode through the reference electrical contact, Rₛₕₑₑₜ is the predetermined resistivity per unit area of the electrode testing surface, r is the distance between the electrode and the reference electrical contact, and r₀ is the radius of electrode touching contact area between the sheet and the electrode, and “ln” represents the natural logarithm.

This relation may provide a direct calculation for the distance between the electrodes in accordance with:

$$r = {r}_{0} \times {e}^{\frac{2 π}{{R}_{sheet}} R ( r )} .$$

Generally, the one or more processors 530, exemplified in FIG. 2, may be configured and operable to determine relative distance between electrodes, location of electrodes with respect to reference electric contacts, and determine spatial mapping of the electrodes by triangulation as described herein. In some examples, the processor may be configured and operable to transmit data on determined impedance or resistance values to a remote processing unit for spatial mapping of the electrodes.

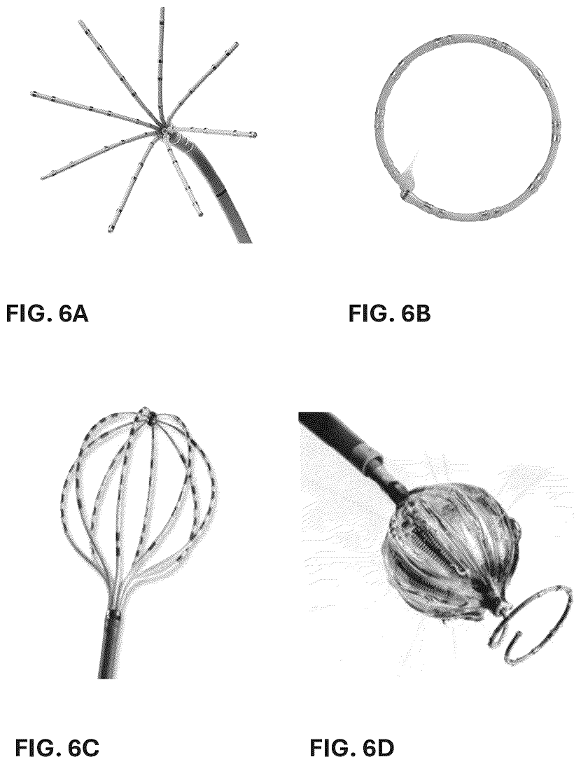

The use of an electrode testing surface enables testing of various types of catheters including different arrangements of electrodes at the distal ends thereof. FIGS. 6A to 6D exemplify different catheter configurations that may be tested using the technique described herein. Moreover, mapping the spatial arrangement of the electrodes enables the technique of the present disclosure to provide output data indicative of electrical continuity as well as spatial structure of the catheter. This may enable detecting electrical issues as well as structural deformation of the catheter. For example, FIG. 6A shows a catheter having a distal end including multiple strands, where each strand carries a number of electrodes. Such catheter can be tested on a planar electrode testing surface by spreading the strands. Determining spatial arrangement of the electrodes enables identifying the structure of the distal end of the catheter for deformation, breaking of the strands, or movement of electrodes along the strands. FIG. 6B exemplifies a catheter having a circular distal end, which can also be tested using a planar electrode testing surface.

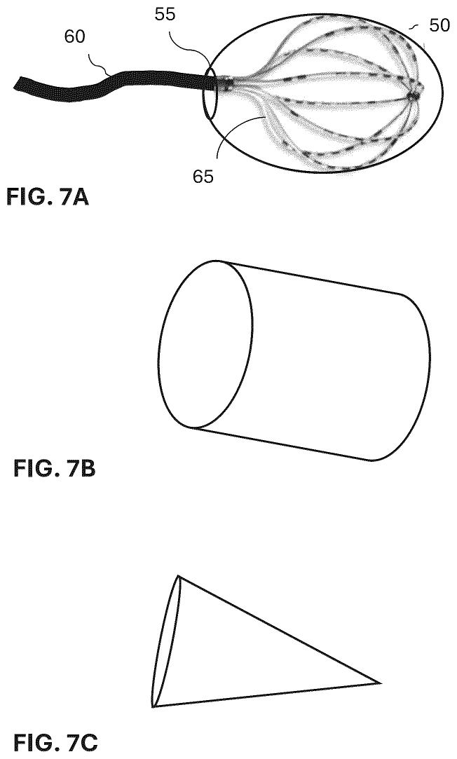

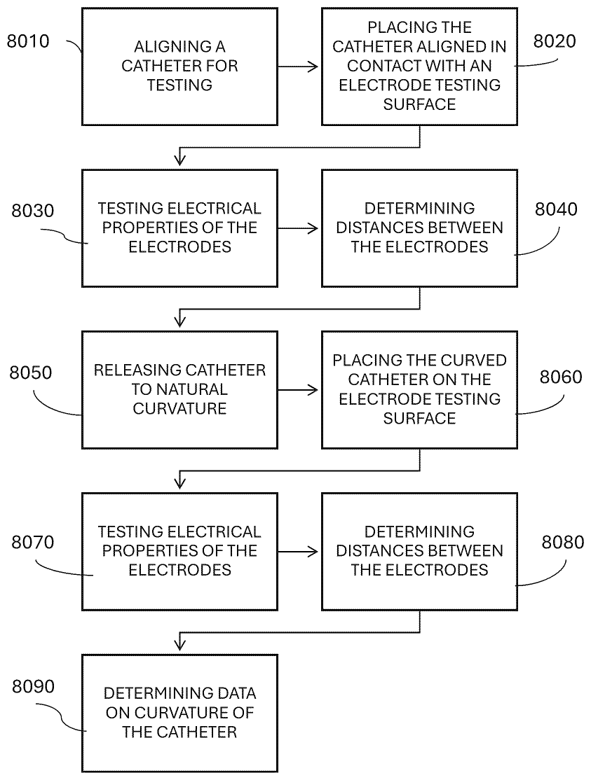

As mentioned above, the technique of the present disclosure enables testing of catheters having various shapes of the distal ends thereof. FIGS. 6C and 6D show catheters whose distal ends have a three-dimensional structure. FIG. 6C shows a catheter having an arrangement of strands that connect at a far end thereof, and FIG. 6D shows a complex catheter shape having a balloon-like structure and further strand extending therefrom. According to some examples of the present disclosure, the electrode testing surface may be rolled or folded into a selected shape, enabling direct testing of such catheters and others. This is exemplified in FIGS. 7A to 7C exemplifying various configurations of the electrode testing surface, suitable for testing of different catheter types. FIG. 7A exemplifies the electrode testing surface 110 configured as an ellipsoid closed surface, having at least one opening 55. While the electrode testing surface is exemplified in FIG. 7A as having an ellipsoid shape, the electrode testing surface 110 may be configured in a spherical shape or other generally closed shapes. The distal end 28 of a catheter 14 is inserted into the close region defined by the electrode testing surface 110, and opened to provide that the different parts of the distal end are in contact with the surface of the sheet. This may utilize a similar mechanism allowing the catheter to be inserted into cavities within a human body, and allowing the distal end to open at a desired location to perform one or more desired functions. Further, FIGS. 7B and 7C exemplify additional closed configurations of the electrode testing surface, being rolled to form a cylinder shape (FIG. 7B) or rolled into a cone shape (FIG. 7C).

The different configurations of the electrode testing surface, and general flexibility of the sheet, enabling it to be shaped in a general free-from configuration, allow for inspection of various catheter structures. As indicated above, the electrode testing surface may be used to provide an electrically conductive surface, having predetermined, and preferably known surface impedance (typically surface resistivity). When in contact with the electrodes of the catheter, the electrode testing surface allows for closing one or more electric circuits using different electrodes, and monitor electrical conductivity through the electrodes. Further, utilizing the predetermined surface impedance of the electrode testing surface enables determining, or at least estimating, a distance between pairs of electrodes in accordance with total impedance between them, and therefore allows to determine spatial mapping of the electrodes.

Further, in some examples, the technique of the present disclosure enables determining data on curvature or natural path defined by the catheter. FIG. 8 exemplifies a method for testing a catheter including determining curvature thereof according to some examples of the present disclosure. The method includes aligning the catheter (step 8010) to a straight-line configuration, placing the aligned catheter on the electrode testing surface (step 8020), and testing the electrodes when the catheter is in a straight line configuration (step 8030). While testing the electrodes according to examples of the present disclosure provides data on electrical conductivity of the electrodes, the technique may also enable determining distances between the electrodes on the electrode testing surface in accordance with impedance between the electrodes and surface resistivity of the surface. Thus, as indicated above, testing of a catheter according to the present technique enables determining distances between the electrodes on the catheter (step 8040). When the catheter is aligned to a straight line, this indicates positions of the electrodes on the catheter. The straight-line testing can be followed by (or performed after) testing of the electrodes in natural curvature of the catheter. Accordingly, the method includes releasing the catheter to natural curvature (step 8050), placing the curved catheter on the electrode testing surface (8060) and testing the electrodes for electrical properties (step 8070). Based on variations in impedance measured between the electrodes, the spatial arrangement, and distances between the electrodes can be estimated again (step 8080), enabling to determine data on curvature of the catheter (step 8090) indicating that the catheter has its intended shape and curvature, and can be used, or if it is faulty or misshaped.

### EXAMPLES

Example 1: A method for testing a catheter (14) carrying an electrode arrangement (70), the method comprising:
(a) bringing (2010) a portion of the catheter (14) having electrodes thereon into contact with an electrode testing surface (110) having a predetermined impedance per unit area;
(b) performing electrical impedance mapping of the electrode arrangement (2020), the electrical impedance mapping comprising:
   selecting (3010) a pair of electrodes (26) comprising at least one electrode of the electrode arrangement (70);
   closing an electrical circuit (3020) between the selected pair of electrodes (26);
   determining an impedance value of the electrical circuit (3040);
   determining electrical continuity of the pair of electrodes and a distance between the pair of electrodes on the electrode testing surface based on the impedance value (3050);
(c) providing output data indicating electrical continuity and spatial mapping of the electrode arrangement based on the electrical impedance mapping.

Example 2: The method of example 1, wherein the electrode testing surface (110) is a polymeric surface/sheet with a surface resistivity of 50 Ohm/square or greater.

Example 3: The method of example 1 or 2, wherein the catheter (14) comprises one or more strands and having one or more electrodes (26) along the one or more strands, and wherein the electrode testing surface (110) is planar, the method further comprising spreading the one or more strands in a star formation on the electrode testing surface (110) to establish electrical contact between electrodes (26) and the electrode testing surface (110).

Example 4: The method of any one of examples 1 to 3, wherein the electrode testing surface (110) forms a curved surface conforming to the spatial structure of the distal end (28) of the catheter (14). The curved surface may be selected from the group comprising of: a cylinder, a portion of a cone, a portion of a sphere, and a portion of an ellipsoid.

Example 5: The method of any one of examples 1 to 4, wherein determining a distance between the pair of electrodes (26) on the electrode testing surface (110) based on the impedance value comprises determining a value l satisfying the relation:

$$l = \frac{R \times W}{{R}_{sheet}}$$

where R is the impedance value, Rₛₕₑₑₜ is the predetermined impedance per unit area of the electrode testing surface (110), and W is the width of electrode testing surface (110).

Example 6: The method of any one of examples 1 to 5, further comprising determining data on a curvature of the catheter (14), comprising:
aligning the catheter (14) and testing the aligned catheter (14) using the electrode testing surface (110);
determining distances between electrodes (26) on the aligned catheter (14);
releasing the catheter (14) and testing the catheter (14) in natural curvature using the electrode testing surface (110);
determining distances between electrodes (26) on the catheter in natural curvature;
using distances between the electrodes on the aligned catheter and distances between the electrodes in natural curvature and determining data on curvature of the catheter (14).

Example 7: The method of any one of examples 1 to 6, further comprising providing at least one reference electrical contact on the electrode testing surface (110), the electrical impedance mapping comprising:
determining impedance values between electrodes of the electrode arrangement and the reference electrical contact; and
determining spatial locations of the electrodes based on the impedance values; wherein determining spatial locations of the electrodes based on the impedance values comprises determining a location (x,y) satisfying the relation:

$$R ( r ) = \frac{{R}_{sheet}}{2 π} \times ln [ \frac{r}{{r}_{0}} ]$$

where R(r) is the impedance value between an electrode and the reference electrical contact, Rₛₕₑₑₜ is the predetermined impedance per unit area of the electrode testing surface, r is the distance between the electrode and the reference electrical contact, and r₀ is the radius of electrode touching contact area between the sheet and the electrode, and “ln” represents the natural logarithm.

Example 8: A system for testing a catheter carrying an electrode arrangement, comprising: an electrical controller circuit (500) configured to connect to the catheter (14) and to the electrode arrangement (70) thereof; and an electrode testing surface (110) having a predetermined impedance per unit area, wherein the electrical controller circuit (500) comprises: an impedance measuring circuit (520) configured to determine impedance values between pairs of electrodes along a surface of the electrode testing surface (110), and processing circuitry (530) configured to utilize the impedance values to determine electrical continuity of the electrodes and assess structure and electrical properties of the catheter (14).

Example 9: The system of example 8, further comprising instructions for an operator to bring the distal portion of the catheter into contact with the electrode testing surface to establish contact between the electrodes and the electrode testing surface.

Example 10: The system of example 8 or 9, wherein the electrode testing surface (110) is planar, and the instructions include spreading strands of the distal end (28) of the catheter (14) in a star formation on the electrode testing surface (110) to establish electrical contact.

Example 11: The system of example 8, wherein the electrode testing surface forms a curved surface conforming to the spatial structure of the distal end of the catheter. The curved surface is selected from the group comprising: a cylinder, a portion of a cone, a portion of a sphere, and a portion of an ellipsoid.

Example 12: The system of any one of examples 8 to 11, wherein the electrode testing surface is a polymeric surface/sheet with a surface resistivity of 50 Ω/square or greater. The electrode testing surface may have surface resistivity of 100 Ω/square or greater, or 200 Ω/square or greater, or 300 Ω/square or greater, of 400 Ω/square or greater, or of 500 Ω/square or greater.

Example 13: The system of any one of examples 8 to 12, wherein the processing circuitry (500) is further configured to determine a spatial map of electrodes (26) of the electrode arrangement (70) by determining distances between electrodes based on impedance values. In some examples, determining the distance between the pairs of electrodes (26) on the electrode testing surface (110) based on the impedance values comprises determining a value l satisfying the relation:

$$l = \frac{R \times W}{{R}_{sheet}}$$

where R is the impedance value, Rₛₕₑₑₜ is the predetermined impedance per unit area of the electrode testing surface (110), and W is the width of the electrode testing surface (110).

Example 14: The system of any one of examples 8 to 13, further comprising at least one reference electrical contact (80) on the electrode testing surface (110), wherein the electrical impedance mapping comprises:
determining impedance values between electrodes (26) of the electrode arrangement (70) and the reference electrical contact (80); and determining spatial locations of the electrodes (26) based on the impedance values.

Example 15: The system of example 14, wherein determining spatial locations of the electrodes (26) based on the impedance values comprises determining a location (x,y) satisfying the relation:

$$R ( r ) = \frac{{R}_{sheet}}{2 π} \times ln [ \frac{r}{{r}_{0}} ]$$

where R(x,y) is the impedance value between an electrode and the reference electrical contact (80), Rₛₕₑₑₜ is the predetermined impedance per unit area of the electrode testing surface (110), r is the distance between the electrode and the reference electrical contact, and r₀ is the radius of electrode touching contact area between the sheet and the electrode, and “ln” represents the natural logarithm.

Example 16: A method for testing a catheter (14) carrying an electrode arrangement (70), the method comprising:
(a) bringing (2010) a portion of the catheter (14) having electrodes thereon into contact with an electrode testing surface (110) having a predetermined impedance per unit area;
(b) performing electrical impedance mapping of the electrode arrangement (2020), the electrical impedance mapping comprising:
   selecting (3010) a pair of electrodes (26) comprising at least one electrode of the electrode arrangement (70);
   closing an electrical circuit (3020) between the selected pair of electrodes (26);
   determining an impedance value of the electrical circuit (3040);
   determining electrical continuity of the pair of electrodes and a distance between the pair of electrodes on the electrode testing surface based on the impedance value (3050);
(c) providing output data indicating electrical continuity and spatial mapping of the electrode arrangement based on the electrical impedance mapping.

Example 17: The method of example 16, wherein the catheter (14) comprises one or more strands and having one or more electrodes (26) along the one or more strands, and wherein the electrode testing surface (110) is planar, the method further comprising spreading the one or more strands in a star formation on the electrode testing surface (110) to establish electrical contact between electrodes (26) and the electrode testing surface (110).

Example 18: The method of example 16, wherein the electrode testing surface (110) forms a curved surface conforming to the spatial structure of the distal end (28) of the catheter (14). The curved surface may be selected from the group comprising of: a cylinder, a portion of a cone, a portion of a sphere, and a portion of an ellipsoid.

Example 19: The method of example 16, wherein determining a distance between the pair of electrodes (26) on the electrode testing surface (110) based on the impedance value comprises determining a value l satisfying the relation:

$$l = \frac{R \times W}{{R}_{sheet}}$$

where R is the impedance value, Rₛₕₑₑₜ is the predetermined impedance per unit area of the electrode testing surface (110), and W is the width of electrode testing surface (110).

Example 20: The method of example 16, further comprising providing at least one reference electrical contact on the electrode testing surface (110), the electrical impedance mapping comprising:
determining impedance values between electrodes of the electrode arrangement and the reference electrical contact; and
determining spatial locations of the electrodes based on the impedance values; wherein determining spatial locations of the electrodes based on the impedance values comprises determining a location (x,y) satisfying the relation:

$$R ( r ) = \frac{{R}_{sheet}}{2 π} \times ln [ \frac{r}{{r}_{0}} ]$$

where R(r) is the impedance value between an electrode and the reference electrical contact, Rₛₕₑₑₜ is the predetermined impedance per unit area of the electrode testing surface, r is the distance between the electrode and the reference electrical contact, and r₀ is the radius of electrode touching contact area between the sheet and the electrode, and “ln” represents the natural logarithm.

Example 21: A system for testing a catheter carrying an electrode arrangement, comprising: an electrical controller circuit (500) configured to connect to the catheter (14) and to the electrode arrangement (70) thereof; and an electrode testing surface (110) having a predetermined impedance per unit area, wherein the electrical controller circuit (500) comprises: an impedance measuring circuit (520) configured to determine impedance values between pairs of electrodes along a surface of the electrode testing surface (110), and processing circuitry (530) configured to utilize the impedance values to determine electrical continuity of the electrodes and assess structure and electrical properties of the catheter (14).

Example 22: The system of example 21, further comprising instructions for an operator to bring the distal portion of the catheter into contact with the electrode testing surface to establish contact between the electrodes and the electrode testing surface.

Example 23: The system of example 21, wherein the electrode testing surface (110) is planar, and the instructions include spreading strands of the distal end (28) of the catheter (14) in a star formation on the electrode testing surface (110) to establish electrical contact.

Example 24: The system of example 21, wherein the electrode testing surface forms a curved surface conforming to the spatial structure of the distal end of the catheter. The curved surface is selected from the group comprising: a cylinder, a portion of a cone, a portion of a sphere, and a portion of an ellipsoid.

Example 25: The system of examples 21, wherein the electrode testing surface is a polymeric surface/sheet with a surface resistivity of 50 Ω/square or greater. The electrode testing surface may have surface resistivity of 100 Ω/square or greater, or 200 Ω/square or greater, or 300 Ω/square or greater, of 400 Ω/square or greater, or of 500 Ω/square or greater.

It is to be noted that the various features described in the various examples can be combined according to all possible technical combinations.

It is to be understood that the invention is not limited in its application to the details set forth in the description contained herein or illustrated in the drawings. The invention is capable of other examples and of being practiced and carried out in various ways. Hence, it is to be understood that the phraseology and terminology employed herein are for the purpose of description and should not be regarded as limiting. As such, those skilled in the art will appreciate that the conception upon which this disclosure is based can readily be utilized as a basis for designing other structures, methods, and systems for carrying out the several purposes of the presently disclosed subject matter.

Those skilled in the art will readily appreciate that various modifications and changes can be applied to the examples of the invention as hereinbefore described without departing from its scope, defined in and by the appended claims.

1. A method for testing a catheter carrying an electrode arrangement, the method comprising:
(a) bringing a portion of the catheter having electrodes thereon, into contact with an electrode testing surface having a predetermined impedance per unit area;
(b) performing electrical impedance mapping of the electrode arrangement, by:
selecting a pair of electrodes comprising at least one electrode of the electrode arrangement;
closing an electrical circuit between the selected pair of electrodes;
determining an impedance value of the electrical circuit;
determining electrical continuity of the pair of electrodes and a distance between the pair of electrodes on the electrode testing surface based on the impedance value;
(c) providing output data indicating electrical continuity and spatial mapping of the electrode arrangement based on the electrical impedance mapping.

2. The method of claim 1, wherein the electrode testing surface is a polymeric surface/sheet with a surface resistivity of 50 Ohm/square or greater.

3. The method of claim 1, wherein the catheter comprises one or more strands and having one or more electrodes along the one or more strands, and wherein the electrode testing surface is planar, the method further comprising spreading the one or more strands in a star formation on the electrode testing surface to establish electrical contact between electrodes and the electrode testing surface.

4. The method of claim 1, wherein the electrode testing surface forms a curved surface conforming to a spatial structure of a distal end of the catheter.

5. The method of claim 4, wherein the curved surface is selected from the group consisting of: a cylinder, a portion of a cone, a portion of a sphere, and a portion of an ellipsoid.

6. The method of claim 1, wherein determining a distance between the pair of electrodes on the electrode testing surface based on the impedance value comprises determining a value l satisfying the relation:
$$l = \frac{R \times W}{{R}_{sheet}}$$
where R is the impedance value, Rₛₕₑₑₜ is the predetermined impedance per unit area of the electrode testing surface, and W is the width of the electrode testing surface.

7. The method of claim 6, further comprising determining data on a curvature of the catheter, comprising:
aligning the catheter and testing the aligned catheter;
determining distances between electrodes on the aligned catheter;
releasing the catheter and testing the catheter in natural curvature;
determining distances between electrodes on the catheter in natural curvature;
using distances between the electrodes on the aligned catheter and distances between the electrodes in natural curvature and determining data on curvature of the catheter.

8. The method of claim 1, further comprising providing at least one reference electrical contact on the electrode testing surface, the electrical impedance mapping comprising:
determining impedance values between electrodes of the electrode arrangement and the reference electrical contact; and
determining spatial locations of the electrodes based on the impedance values.

9. The method of claim 8, wherein determining spatial locations of the electrodes based on the impedance values comprises determining a location (x,y) satisfying the relation:
$$R ( r ) = \frac{{R}_{sheet}}{2 π} \times ln [ \frac{r}{{r}_{0}} ]$$
where R(x,y) is the impedance value between an electrode and the reference electrical contact, Rₛₕₑₑₜ is the predetermined impedance per unit area of the electrode testing surface, r is the distance between the electrode and the reference electrical contact, and r₀ is the radius of electrode touching contact area between the sheet and the electrode, and “ln” represents the natural logarithm.

10. A system for testing a catheter carrying an electrode arrangement, comprising:
an electrical controller circuit configured to connect to the catheter and to the electrode arrangement thereof; and
an electrode testing surface having a predetermined impedance per unit area,
wherein the electrical controller circuit comprises:
an impedance measuring circuit configured to determine impedance values between pairs of electrodes along a surface of the electrode testing surface, and
processing circuitry configured to utilize the impedance values to determine electrical continuity of the electrodes and assess structure and electrical properties of the catheter.

11. The system of claim 10, further comprising instructions for an operator to bring a distal end of the catheter into contact with the electrode testing surface to establish contact between the electrodes and the electrode testing surface.

12. The system of claim 11, wherein the electrode testing surface is planar, and the instructions include spreading strands of the distal end of the catheter in a star formation on the electrode testing surface to establish electrical contact.

13. The system of claim 10, wherein the electrode testing surface forms a curved surface conforming to the spatial structure of the distal end of the catheter.

14. The system of claim 13, wherein the curved surface is selected from the group consisting of: a cylinder, a portion of a cone, a portion of a sphere, and a portion of an ellipsoid.

15. The system of claim 10, wherein the electrode testing surface is a polymeric surface/sheet with a surface resistivity of 500 Ohm/square or greater.

16. The system of claim 10, wherein the processing circuitry is further configured to determine a spatial map of electrodes of the electrode arrangement by determining distances between electrodes based on impedance values.

17. The system of claim 16, wherein determining a distance between the pairs of electrodes on the electrode testing surface based on the impedance values comprises determining a value l satisfying the relation:
$$l = \frac{R \times W}{{R}_{sheet}}$$
where R is the impedance value, Rₛₕₑₑₜ is the predetermined impedance per unit area of the electrode testing surface, and W is the width of the electrode testing surface.

18. The system of claim 16, further comprising at least one reference electrical contact on the electrode testing surface, wherein the electrical impedance mapping comprises:
determining impedance values between electrodes of the electrode arrangement and the reference electrical contact; and
determining spatial locations of the electrodes based on the impedance values.

19. The system of claim 18, wherein determining spatial locations of the electrodes based on the impedance values comprises determining a location (x,y) satisfying the relation:
$$R ( r ) = \frac{{R}_{sheet}}{2 π} \times ln [ \frac{r}{{r}_{0}} ]$$
where R(x,y) is the impedance value between an electrode and the reference electrical contact, Rₛₕₑₑₜ is the predetermined impedance per unit area of the electrode testing surface, r is the distance between the electrode and the reference electrical contact, and r₀ is the radius of electrode touching contact area between the sheet and the electrode, and “ln” represents the natural logarithm.
